(19) United States
(12) Patent Application Publication
(10) Pub. No.: US 2007/0232658 A1
(43) Pub. Date: Oct. 4, 2007

(54) Protein Kinase C Inhibitors for the Treatment of Autoimmune Diseases and of Transplant Rejiction

(76) Inventors: Jurgen Wagner, Bottmingen (CH); Walter Schuler, Grenzach-Wyhlen (DE)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/599,697

(22) PCT Filed: Apr. 7, 2005
(86) PCT No.: PCT/EP05/03663

§ 371 Date: Oct. 5, 2006

(30) Foreign Application Priority Data
Apr. 8, 2004 (GB) 0408066.9
Jun. 29, 2004 (GB) 0414540.5
Oct. 5, 2004 (GB) 0422068.7

(51) Int. Cl. A61K 31/454; A61K 31/4015; A61P 11/06; A61P 19/02; A61P 37/06; A61P 21/04; A61P 17/06; A61K 31/404

(52) U.S. Cl. 514/323; 514/411; 514/418

(57) ABSTRACT

This invention relates to the use of a compound of formula I, II, III, or IV, as described in the specification, in transplantation and autoimmune diseases.

The present invention relates to new uses of protein kinase C inhibitors.

In particular, the present invention relates to new uses of protein kinase C inhibitors of formula I, II, III and IV and pharmaceutically acceptable salts, hydrates or solvates thereof.

Protein kinase C inhibitors of formula I are as follows: 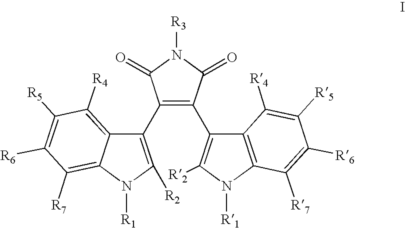
wherein
each of R₁ and R′₁, independently, is hydrogen, alkyl, haloalkyl, alkenyl, arylalkyl, alkoxyalkyl, hydroxyalkyl, aminoalkyl, monoalkylaminoalkyl, dialkylaminoalkyl, acylaminoalkyl, acyloxyalkyl, cyanoalkyl, amidinoalkyl, carboxyalkyl, alkoxycarbonylalkyl, aminocarbonylalkyl, or a group of the formula (a), (b) or (c) 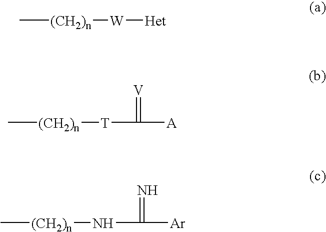
   wherein Het signifies a heterocyclyl group; W signifies NH, S or a bond; T signifies NH or S; V signifies O, S, NH, or NCN; A signifies alkylthio, amino, monoalkylamino or dialkylamino; Ar signifies aryl;
each of R₂ and R′₂, independently, is hydrogen, alkyl, alkoxyalkyl, hydroxyalkyl, C₁-C₃alkylthio, S(O)C₁-C₃alkyl, CF₃;
or R₁ and R₂ form together —(CH₂)ᵣ—X—CH₂— wherein r is 1, 2, or 3, and X is CHR₈ or NR₈ wherein R₈ is (CH₂)ₛR₉ wherein R₉ is hydrogen, hydroxy, alkoxy, amino, monoalkylamino, dialkylamino, trialkylamino, azido, acylamino, alkoxycarbonyl, cyano, amidino, or aminocarbonyl, and s is 0, 1, 2 or 3;
R₃ is hydrogen or CH₃CO;
each of R₄, R′₄, R₅, R′₅, R₆, R′₆, R₇ and R′₇, independently, is hydrogen, halogen, alkyl, hydroxy, alkoxy, —COO(C₁-C₃alkyl), CF₃, nitro, amino, acetylamino, monoalkylamino, dialkylamino, alkylthio, C₁-C₃alkylthio, or S(O)C₁-C₃alkyl; and
n is 1, 2, 3, 4, 5 or 6.

Protein kinase C inhibitors of formula II are as follows: 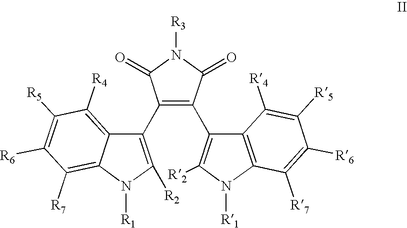
wherein
R₁ is a group of formula (d), (e) or (f) 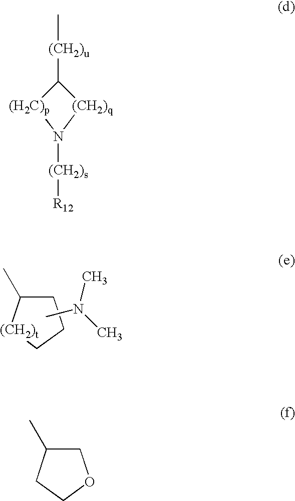
   wherein each of p and q independently is 1, 2, 3, or 4;
   s is 0, 1, 2 or 3;
   t is 1 or 2;
   u is 0 or 1; and
   R₁₂ is hydrogen, alkyl, haloalkyl, cycloalkyl, acetyl, aryl, —CH(aryl)₂, amino, monoalkylamino, dialkylamino, guanidino, —C(═N(alkoxycarbonyl))NH(alkyoxycarbonyl), amidino, hydroxy, carboxy, alkoxycarbonyl or heterocyclyl;
R′₁ is hydrogen, C₁₋₄alkyl, aminoalkyl, monoalkylaminoalkyl, or dialkylaminoalkyl, each of R₂ and R′₂, independently, is hydrogen, alkyl, alkoxyalkyl, hydroxyalkyl, C₁-C₃alkylthio, S(O)C₁-C₃alkyl, CF₃;
R₃ is hydrogen or CH₃CO—; and
each of R₄, R′₄, R₅, R′₅, R₆, R′₆, R₇ and R′₇, independently, is hydrogen, halogen, alkyl, hydroxy, alkoxy, —COO(C₁-C₃alkyl), CF₃, nitro, amino, acetylamino, monoalkylamino, dialkylamino, alkylthio, C₁-C₃alkylthio, or S(O)C₁-C₃alkyl.

Protein kinase C inhibitors of formula III are as follows: 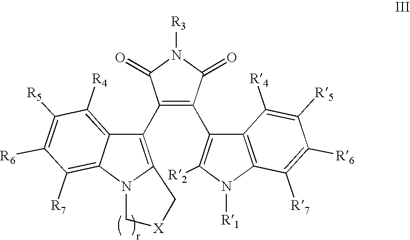
wherein
R′₁ is hydrogen, C₁-C₄alkyl, aminoalkyl, monoalkylaminoalkyl, or dialkylaminoalkyl;
R′₂ is hydrogen, alkyl, alkoxyalkyl, hydroxyalkyl, C₁-C₃alkylthio, S(O)C₁-C₃alkyl, CF₃ R₃ is hydrogen or CH₃CO—;
each of R₄, R′₄, R₅, R′₅, R₆, R′₆, R₇ and R′₇, independently, is hydrogen, halogen, alkyl, hydroxy, alkoxy, —COO(C₁-C₃alkyl), CF₃, nitro, amino, acetylamino, monoalkylamino, dialkylamino, alkylthio, C₁-C₃alkylthio, or S(O)C₁-C₃alkyl;
X is CR₈R₉ wherein R₈ is (CH₂)ₛR₁₀ wherein R₉ is (CH₂)ₛR₁₁, each of R₁₀ and R₁₁, independently, is hydroxy, alkoxy, carboxy, acyloxy, amino, monoalkylamino, dialkylamino, trialkylamino, azido, acylamino, alkoxycarbonyl, cyano, amidino, or aminocarbonyl, and s is 0, 1, 2 or 3; and
r is 1, 2, or 3.

Protein kinase C inhibitors of formula IV are as follows: 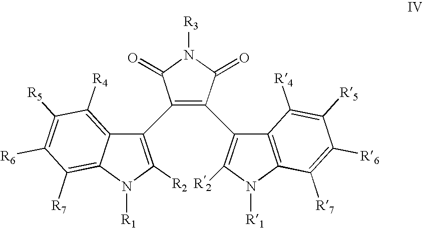
wherein
R₁ is alkylglycose residue or a group of formula (g) or (h) 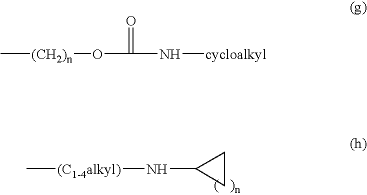
   wherein n is 1, 2, 3, 4, 5 or 6;
R′₁ is hydrogen, C₁-C₄alkyl, cyclopropylmethyl, aminoalkyl, monoalkylaminoalkyl, or dialkylaminoalkyl;
each of R₂ and R′₂, independently, is hydrogen, alkyl, alkoxyalkyl, hydroxyalkyl, C₁-C₃alkylthio, S(O)C₁-C₃alkyl, CF₃;
R₃ is hydrogen or CH₃CO—; and
each of R₄, R′₄, R₅, R′₅, R₆, R′₆, R₇ and R′₇, independently, is hydrogen, halogen, alkyl, hydroxy, alkoxy, —COO(C₁-C₃alkyl), CF₃, nitro, amino, acetylamino, monoalkylamino, dialkylamino, alkylthio, C₁-C₃alkylthio, or S(O)C₁-C₃alkyl.

Alkyl, alone or in combinations, may be a straight or branched-chain alkyl group containing from 1 to 7, preferably 1 to 4, carbon atoms such as methyl, ethyl, propyl, isopropyl, butyl, sec-butyl, t-butyl and pentyl. “C₁-C₃alkyl” is an alkyl limited to one to four carbon atoms. Alkenyl may be a 2 to 7 carbon, straight or branched hydrocarbon containing one or more double bonds, preferably one or two double bonds. Examples of alkenyl include ethenylene, propenylene, 1,3 butadienyl, and 1,3,5-hexatrienyl.

Cycloalkyl, alone or in combinations, may be a 3 to 7 carbon cycloalkyl, e.g. cyclopropyl, cyclobutyl, cyclopentyl, cyclohexyl, and cycloheptyl.

Alkoxy, alone or in combinations, may be an alkyl covalently bonded by an —O— linkage. Examples of alkoxy groups include methoxy, ethoxy, propoxy, isopropoxy, butoxy and t-butoxy. Alkoxyalkyl may be e.g. CH₃(CH₂)—O—(CH₂)ₘ may be e.g. t-butoxycarbonyl or BOC.

Haloalkyl may be an alkyl with one or more, preferably 1 to 3 halogen atoms, e.g. CH₂Cl, CF₃, CH₂CF₃, CH₂(CF₂)₂CF₃, and the like.

The acyl moiety of an acylamino or acylaminoalkyl group is derived from an alkanoic acid containing a maximum of 7, preferably a maximum of 4, carbon atoms, e.g. acetyl, propionyl or butyryl, or from an aromatic carboxylic acid, e.g. benzoyl. An acyloxy is one such acyl bonded by an —O— linkage e.g. acetyloxy, CH₃C(═O)O—. An acylamino is e.g. CH₃(C═O)NH—(acetylamino). Likewise, an acylaminoalkyl is CH₃ (C═O)NH(CH₂)ₘ—.

Aryl, alone or in combinations, may be an unsubstituted phenyl group or a phenyl group carrying one or more, preferably 1 to 3, substituents, independently selected from halogen, alkyl, hydroxy, benzyloxy, alkoxy, haloalkyl, nitro, amino, acylamino, monoalkylamino, dialkylamino, alkylthio, alkylsulfinyl, alkylsulfonyl and cyano. Arylalkyl is preferably benzyl.

Halogen may be fluorine, chlorine, bromine or iodine.

The heterocyclic group denoted by “Het” or “heterocyclyl” may be a stable, saturated, partially unsaturated, or aromatic 5- or 6-membered heterocyclic group. The heterocyclic ring consists of carbon atoms and from 1 to 3 heteroatoms independently selected from the group consisting of nitrogen, oxygen, and sulfur. The heterocyclic group may optionally be substituted with 1 to 3 substituents independently selected from halogen, alkyl, hydroxy, alkoxy, haloalkyl, nitro, amino, acylamino, monoalkylamino, dialkylamino, alkylthio, alkylsulfinyl and alkylsulfonyl or, when the heterocyclyl group is an aromatic nitrogen-containing heterocyclic group, the nitrogen atom can carry an oxide group. Examples of such heterocyclyl groups include imidazolyl, imidazolinyl, thiazolinyl, pyridyl, indolyl, furyl, and pyrimidinyl.

“Alkylglycose residue” may be a glycose moiety linked in the C-1 position to the indolyl via a C₂-C₄alkyl. Glycoses included in alkylglycose residue are natural or unnatural 5 or 6 carbon sugars, preferably selected from allosyl, altrosyl, glucosyl, mannosyl, gulosyl, idosyl, galactosyl, talosyl, arabinosyl, xylosyl, lyxosyl, rhamnosyl, ribosyl, deoxyfuranosyl, deoxypyranosyl, and deoxyribosyl. The glycose may be azide substituted, O-acetylated, O-methylated, amino, mono- and di-alkylamino substituted, or acylamino substituted. For example, alkylglycose residue includes

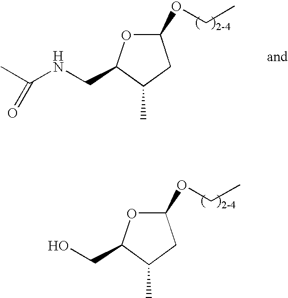

The compounds of formulae I to IV may exist in free form or in salt form, e.g. addition salts with e.g. an organic or inorganic acid, for example, hydrochloride, phosphate, acetate, mesylate, citrate or tartrate. The compounds of formulae I to IV in free form or in salt form may be used according to the invention in hydrate or solvate forms, in amorphous or crystalline form.

Particularly preferred protein kinase C inhibitors are compounds of formula Ia, Ib, IIa and IIIa or a salt thereof.

Compounds of formula Ia are as follows 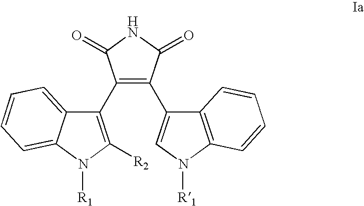
wherein
R₁ is hydrogen, aminoalkyl, monoalkylaminoalkyl, or dialkylaminoalkyl;
R′₁ is hydrogen, C₁₋₄alkyl, aminoalkyl, monoalkylaminoalkyl, or dialkylaminoalkyl; and
R₂ is hydrogen or methyl.

Compounds of formula Ib are as follows 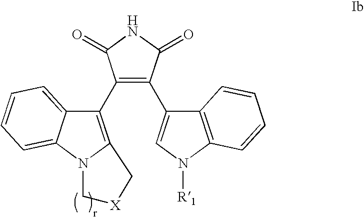
wherein
R′₁ is hydrogen, or C₁-C₄alkyl;
X is CR₈R₉ or NR₈ wherein R₈ is (CH₂)ₛR₁₀ wherein R₉ is (CH₂)ₛR₁₁, each of R₁₀ and R₁₁, independently, is hydrogen, hydroxy, amino, monoalkylamino, or dialkylamino, and s is 1; and
r is 1 or 2.

Compounds of formula IIa are as follows 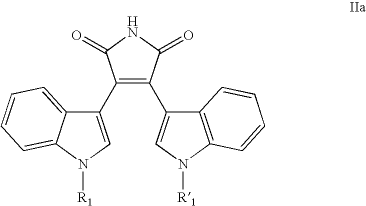
wherein
R₁ is 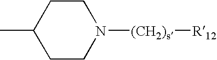
   wherein either s′ is 0 and R′₁₂ is hydrogen or C₁₋₄alkyl; or s′ is 1 and R′₁₂ is pyridyl, preferably 2-pyridyl, and
R′₁ is hydrogen, C₁₋₄alkyl or;

Compounds of formula IIIa are as follows: 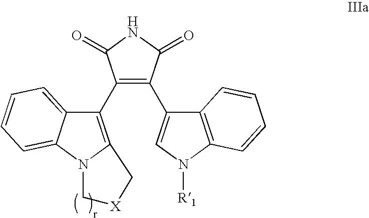
wherein
R′₁ is hydrogen, alkyl, aminoalkyl, monoalkylaminoalkyl, or dialkylaminoalkyl;
X is CR₈R₉ or NR₈ wherein R₈ is (CH₂)ₛR₁₀ wherein R₉ is (CH₂)ₛR₁₁, each of R₁₀ and R₁₁, independently, is hydroxy, carboxy, alkoxycarbonyl, amino, monoalkylamino, or dialkylamino, and s is 0 or 1; and
r is 1 or 2.

Even more preferred are 3-(1-methyl-1H-indol-3-yl)-4-[1-{(1-pyridin-2-ylmethyl)-piperidin-4-yl}-1H-indol-3-yl]-pyrrole-2,5-dione, also called LY 317615 (Compound A hereinafter), and 3-(1-methyl-1H-indol-3-yl)-4-[1-(piperidin-4-yl)-1H-indol-3-yl]-pyrrole-2,5-dione (Compound B hereinafter), in free form or in salt form, e.g. hydrochloride salt.

The compounds of formula I, II, II and IV may be synthesized as known in the art, e.g. as described in U.S. Pat. No. 5,545,636.

Protein kinase C inhibitors of formula I, II, III or IV and pharmaceutically acceptable salts, hydrates or solvates thereof have, on the basis of observed activity, e.g. inhibiting protein kinase C β-1 and β-2 isozymes, e.g. as described in U.S. Pat. No. 5,545,636, been found to be useful in treating conditions associated with diabetes mellitus and its complications, as well as other diseases associated with an elevation of the β-1 and β-2 isozymes, e.g. ischemia, inflammation, central nervous system disorders, cardiovascular disease, dermatological disease, Alzheimer's disease, and cancer.

It has now been found that protein kinase C inhibitors of formula I, II, III or IV, e.g. of formula Ia, Ib, IIa and IIIa, and pharmaceutically acceptable salts, hydrates or solvates thereof are useful for the treatment and prevention of organ, tissue or cell transplant rejection, e.g. for the treatment of recipients of solid organs or tissues, e.g. heart, lung, combined heart-lung, liver, kidney, pancreatic, skin or corneal transplants, or of cells, e.g. stem cells, or insulin-producing cells, e.g. pancreatic islet cells. They are also indicated for the prevention of graft-versus-host disease, such as following bone marrow transplantation.

In accordance with the particular findings of the present invention, there is provided
1.1 A method for treating organ, tissue or cell transplant rejection, e.g. heart, lung, combined heart-lung, liver, kidney, pancreatic, skin or corneal transplant rejection, and for preventing graft-versus-host disease in a subject in need thereof, comprising administering to said subject a therapeutically effective amount of a protein kinase C inhibitor of formula I, II, III or IV, e.g. of formula Ia, Ib, IIa and IIIa, preferably Compound A or B, or a pharmaceutically acceptable salt, hydrate or solvate thereof.

Furthermore, it has now been found that protein kinase C inhibitors of formula I, II, III or IV, e.g. of formula Ia, Ib, IIa and IIIa, and pharmaceutically acceptable salts or solvates thereof are useful for the treatment and prevention of autoimmune diseases, e.g. sarcoidosis, fibroid lung, idiopathic interstitial pneumonia, obstructive airways disease, including conditions such as asthma, intrinsic asthma, extrinsic asthma, dust asthma, particularly chronic or inveterate asthma (for example late asthma and airway hyperreponsiveness), bronchitis, including bronchial asthma, infantile asthma, allergic rheumatoid arthritis, systemic lupus erythematosus, nephrotic syndrome lupus, Hashimoto's thyroiditis, multiple sclerosis, myasthenia gravis, uveitis, nephrotic syndrome, steroid dependent and steroid-resistant nephrosis, palmoplantar pustulosis, allergic encephalomyelitis, glomerulonephritis, psoriasis, psoriatic arthritis, atopic eczema (atopic dermatitis), contact dermatitis and further eczematous dermatitises, seborrheic dermatitis, lichen planus, pemphigus, bullous pemphigoid, epidermolysis bullosa, urticaria, angioedemas, vasculitides, erythemas, cutaneous eosinophilias, acne, alopecia areata, eosinophilic fasciitis, atherosclerosis, conjunctivitis, keratoconjunctivitis, keratitis, vernal conjunctivitis, uveitis associated with Behcet's disease, herpetic keratitis, conical cornea, dystorphia epithelialis corneae, keratoleukoma, ocular pemphigus, Mooren's ulcer, scleritis, severe intraocular inflammation, inflammation of mucosa or blood vessels such as leukotriene B4-mediated diseases, gastric ulcers, vascular damage caused by ischemic diseases and thrombosis, ischemic bowel disease, inflammatory bowel disease (e.g. Crohn's disease and ulcerative colitis), necrotizing enterocolitis, renal diseases including interstitial nephritis, Goodpasture's syndrome hemolytic uremic syndrome and diabetic nephropathy, nervous diseases selected from multiple myositis, Guillain-Barre syndrome, Meniere's disease and radiculopathy, collagen disease including scleroderma, Wegener's granuloma and Sögren's syndrome, chronic autoimmune liver diseases including autoimmune hepatitis, primary biliary cirrhosis and sclerosing cholangitis), partial liver resection, acute liver necrosis (e.g. necrosis caused by toxins, viral hepatitis, shock or anoxia), B-virus hepatitis, non-A/non-B hepatitis and cirrhosis, fulminant hepatitis, pustular psoriasis, Behcet's disease, active chronic hepatitis, Evans syndrome, pollinosis, idiopathic hypoparathyroidism, Addison disease, autoimmune atrophic gastritis, lupoid hepatitis, tubulointerstitial nephritis, membranous nephritis, amyotrophic lateral sclerosis or rheumatic fever, in particular inflammatory bowel diseases (IBD) such as Crohn's disease and ulcerative colitis; amyotrophic lateral sclerosis (ALS); multiple sclerosis; rheumatoid arthritis or hepatitis C.

Accordingly, the present invention provides
1.2 A method for treating or preventing autoimmune diseases, e.g. as indicated above in a subject in need thereof, comprising administering to said subject a therapeutically effective amount of a protein kinase C inhibitor of formula I, II, III or IV, e.g. of formula Ia, Ib, IIa and IIIa, or a pharmaceutically acceptable salt, hydrate or solvate thereof.

In the present description the terms “treatment” or “treat” refer to both prophylactic or preventive treatment as well as curative or disease modifying treatment, including treatment of patients at risk of contracting the disease or suspected to have contracted the disease as well as patients who are ill or have been diagnosed as suffering from a disease or medical condition.

In a series of further specific or alternative embodiments, the present invention also provides:
2. A protein kinase C inhibitor of formula I, II, III or IV, preferably Compound A or B, or a pharmaceutically acceptable salt, hydrate or solvate thereof for use as an immunosuppressant or immunomodulator, e.g. in the methods as defined above.
3. A protein kinase C inhibitor of formula I, II, III or IV, preferably Compound A or B, or a pharmaceutically acceptable salt, hydrate or solvate thereof for use in the preparation of a pharmaceutical composition for use as an immunosuppressant or immunomodulator, e.g. in the methods as defined above.
4. A pharmaceutical composition for use as an immunosuppressant or immunomodulator, e.g. in the methods as defined above, comprising a protein kinase C inhibitor of formula I, II, III or IV, preferably Compound A or B, or a pharmaceutically acceptable salt, hydrate or solvate thereof together with one or more pharmaceutically acceptable diluents or carriers therefore.

Utility of the compounds of the invention in treating and/or preventing diseases and conditions as hereinabove specified, may be demonstrated in standard animal or clinical tests, e.g. as described hereinafter.

In Vitro: MLR

A murine model MLR, e.g., as described by T. Meo in “Immunological Methods”, L. Lefkovits and B. Peris, Eds., Academic Press, N.Y. pp. 227-239 (1979), is used to demonstrate the immunosuppressive effect of the compounds to be used in the method of the invention. Spleen cells (0.5×10⁶) from Balb/c mice (female, 8-10 weeks) are co-incubated for 5 days with 0.5×10⁶ irradiated (2000 rads) or mitomycin C treated spleen cells from CBA mice (female, 8-10 weeks). The irradiated allogeneic cells induce a proliferative response in the Balb/c spleen cells which can be measured by labeled precursor incorporation into the DNA. Since the stimulator cells are irradiated (or mitomycin C treated) they do not respond to the Balb/c cells with proliferation but do retain their antigenicity. The antiproliferative effect of the compounds on the Balb/c cells is measured at various dilutions and the concentration resulting in 50% inhibition of cell proliferation (IC₅₀) is calculated. Compound B for example has an IC₅₀ of 195 nM.

In Vitro: PKC Assay

The compounds of the invention are tested for their activity on PKC according to the following method. Assay is performed in a white with clear bottom 384-well microtiterplate with non-binding surface. The reaction mixture (25 μl) contains 1.5 μM of a tridecapeptide acceptor substrate that mimics the pseudo substrate sequence of PKC α with the Ala→Ser replacement, 10 μM ³³P-ATP, 10 mM Mg(NO₃)₂, 0.2 mM CaCl₂, PKC at a protein concentration varying from 25 to 400 ng/ml (depending on the isotype used), lipid vesicles (containing 30 mol % phosphatidylserine, 5 mol % DAG and 65 mol % phosphatidylcholine) at a final lipid concentration of 0.5 mM, in 20 mM Tris-HCl buffer pH 7.4+0.1% BSA. Incubation is performed for 60 min at room temperature. Reaction is stopped by adding 50 μl of stop mix (100 mM EDTA, 200 μM ATP, 0.1% Triton X-100, 0.375 mg/well streptavidin-coated SPA beads in phosphate buffered saline w/o Ca, Mg). After 10 min incubation at room temperature, the suspension is spun down for 10 min at 300 g. Incorporated radioactivity is measured in a Trilux counter for 1 min. IC₅₀ measurement is performed on a routine basis by incubating a serial dilution of inhibitor at concentrations ranging between 1-1000 μM. IC₅₀ values are calculated from the graph by curve fitting with XL fit® software.

In this assay, compounds of the invention, e.g. compounds of formula IIa, inhibit PKC with an IC₅₀≦1 μM, preferably ≦10 nM. For example compound B inhibits PKCα with an IC₅₀ of 3.0 nM, PKCβ with an IC₅₀ of 2.0 nM, PKCδ with an IC₅₀ of 2.0 nM, and PKCδ with an IC₅₀ of 2.0 nM.

In Vivo: Rat Heart Transplantation

The strain combination used: Male Lewis (RT¹ haplotype) and DA (RT¹ haplotype). The animals are anaesthetised using inhalational isofluorane. Following heparinisation of the donor rat through the abdominal inferior vena cava with simultaneous exsanguination via the aorta, the chest is opened and the heart rapidly cooled. The aorta is ligated and divided distal to the first branch and the brachiocephalic trunk is divided at the first bifurcation. The left pulmonary artery is ligated and divided and the right side divided but left open. All other vessels are dissected free, ligated and divided and the donor heart is removed into iced saline.

The recipient is prepared by dissection and cross-clamping of the infra-renal abdominal aorta and vena cava. The graft is implanted with end-to-side anastomoses, using 10/0 monofilament suture, between the donor brachiocephalic trunk and the recipient aorta and the donor right pulmonary artery to the recipient vena cava. The clamps are removed, the graft tethered retroabdominally, the abdominal contents washed with warm saline and the animal is closed and allowed to recover under a heating lamp. Graft survival is monitored by daily palpation of the beating donor heart through the abdominal wall. Rejection is considered to be complete when heart beat stops. Increases of graft survival are obtained in animals treated with a compound of formula I, II, III or IV or a pharmaceutically acceptable salt or solvate thereof administered orally at a daily dose of 10 to 30 mg/kg bid. In this model, a prolongation of graft survival for 14, 25, 26 days was obtained with Compound A when administered at a dose of 30 mg/kg bid.

In Vivo: Graft v. Host Model

Spleen cells (2×10⁷) from Wistar/F rats are injected subcutaneously into the right hind footpad of (Wistar/F×Fischer 344)F₁ hybrid rats. The left footpad is left untreated. The animals are treated with the test compounds on 4 consecutive days (0-3). The popliteal lymph nodes are removed on day 7, and the weight differences between two corresponding lymph nodes are determined. The results are expressed as the inhibition of lymph node enlargement (given in percent) comparing the lymph node weight differences in the experimental groups to the weight difference between the corresponding lymph nodes from a group of animals left untreated with a test compound. In this assay, an inhibition of 60 to 80%, preferably 70 to 80%, is obtained with compound A when administered at a dose of 30 mg/kg bid.

In Vivo: Treatment of Multiple Sclerosis: SJL/J Mouse model of chronic progressive experimental autoimmune encephalomyelitis (EAE)

Immunization: On day 0, female SJL/J mice are immunized (subcutaneous flank injection) with 200 μl inoculum containing 500 μg bovine myelin basic protein (MBP) emulsified in complete Freund's adjuvant (CFA). On day 9, mice are boosted by a second MBP injection and an additional intravenous adjuvant injection consisting of 200 ng B. pertussis toxin. A final Pertussis injection is given on day 11.

Most of the MBP-immunized mice exhibit a severe bout of EAE by day 21. This is followed by a recovery phase starting around day 25, during which time mice remain symptom-free for about 20 days. Subsequently, by days 45-47, approximately 50% of the animals go into the progressive phase of the disease. Therefore, therapeutic treatment with test compounds starts on day 21 when the disease is fully established and continues until day 70, unless stated otherwise. Recombinant mouse interferon beta (INFβ Calbiochem/Biosciences) is dissolved in saline and given by intraperitoneal injection 3× per week. Compounds of the invention, e.g. Compound A, are administered p.o. 5× per week by gavage. Mice in the vehicle control group are MBP-immunized and treated with water.

Each experimental group consists of 10 mice, which are examined daily for clinical EAE symptoms. Disease incidence and the day of EAE onset also are recorded. Clinical grades of EAE are assessed using a scale from 0 to 3. Any disease-related mortality which occurs after starting drug treatment is recorded with a maximum score of 3.

Daily dosages required in practicing the method of the present invention will vary depending upon, for example, the compound used, the host, the mode of administration and the severity of the condition to be treated. A preferred daily dosage range is about from 1 mg to about 1000 mg of active substance as a single dose or in divided doses.

Compounds of formula I, II, III or IV, e.g. of formula Ia, Ib, IIa or IIIa, may be administered in free form or in pharmaceutically acceptable salt form, e.g. as indicated above. Such salts may be prepared in conventional manner and exhibit the same order of activity as the free compounds.

Compounds of formula I, II, III or IV, e.g. of formula Ia, Ib, IIa or IIIa, preferably Compound A or Compound B, or pharmaceutically acceptable salts or solvates thereof may be administered as the sole active ingredient or together with other drugs in immunomodulating regimens e.g. for the treatment or prevention of allo- or xenograft acute or chronic rejection or in autoimmune diseases. For example, they may be used in combination with a calcineurin inhibitor, e.g. cyclosporin A, ISA Tx247, FK506, ABT-281, ASM 981; an mTOR inhibitor, e.g. rapamycin, 40-O-(2-hydroxyethyl)-rapamycin, CCI779, ABT578, or a rapalog, e.g. AP23573, AP23464, AP23675, AP23841, TAFA-93, biolimus 7 or biolimus 9 etc.; corticosteroids; cyclophosphamide; azathioprene; methotrexate; an S1P receptor agonist, e.g. FTY 720 or an analogue thereof; leflunomide or analogs thereof; mizoribine; mycophenolic acid or a salt thereof, e.g. sodium salt; mycophenolate mofetil; 15-deoxyspergualine or analogs thereof; immunosuppressive monoclonal antibodies, e.g. monoclonal antibodies to leukocyte receptors, e.g. MHC, CD2, CD3, CD4, CD 11a/CD18, CD7, CD25, CD27, B7, CD40, CD45, CD58, CD 137, ICOS, CD150 (SLAM), OX40, 4-1BB or their ligands, e.g. CD154; or other immunomodulatory compounds, e.g. a recombinant binding molecule having at least a portion of the extracellular domain of CTLA4 or a mutant thereof, e.g. an at least extracellular portion of CTLA4 or a mutant thereof joined to a non-CTLA4 protein sequence, e.g. CTLA4lg (for example designated ATCC 68629) or a mutant thereof, e.g. LEA29Y, or other adhesion molecule inhibitors, e.g. mAbs or low molecular weight inhibitors including LFA-1 antagonists, Selectin antagonists and VLA-4 antagonists.

In accordance with the foregoing the present invention provides in a yet further aspect:
5. A method as defined above comprising co-administration, e.g. concomitantly or in sequence, of a therapeutically effective amount of a protein kinase C inhibitor of formula I, II, III or IV, e.g. of formula Ia, Ib, IIa, or IIIa, e.g. Compound A or Compound B, or a pharmaceutically acceptable salt, hydrate or solvate thereof, and a second drug substance, said second drug substance being an immunosuppressant or immunomodulatory drug, e.g. as indicated above.
6. A therapeutic combination, e.g. a kit, comprising a) a protein kinase C inhibitor of formula I, II, III or IV, e.g. of formula Ia, Ib, IIa, or IIIa, e.g. Compound A or Compound B, or a pharmaceutically acceptable salt, hydrate or solvate thereof, and b) at least one second agent selected from an immunosuppressant and immunomodulatory drug. Component a) and component b) may be used concomitantly or in sequence. The kit may comprise instructions for its administration.

Where a protein kinase C inhibitor of formula I, II, III or IV, e.g. of formula Ia, Ib, IIa, or IIIa, or a pharmaceutically acceptable salt or solvate thereof is administered in conjunction with other immunosuppressant or immunomodulatory drug, e.g. for preventing or treating acute or chronic graft rejection or autoimmune diseases as hereinabove specified, dosages of the co-administered immunosuppressant or immunomodulatory compound will of course vary depending on the type of co-drug employed, e.g. whether it is a steroid or a cyclosporine, on the specific drug employed, on the condition being treated and so forth.

Compound A and Compound B are preferred, particularly for use in the treatment or prevention of graft rejection and for the prevention of the graft versus host disease.

1. A method for treating or preventing organ or tissue transplant rejection or an autoimmune disease or for preventing graft-versus-host disease in a subject in need thereof, comprising administering to said subject a therapeutically effective amount of a protein kinase C inhibitor of formula I, II, III or IV or a pharmaceutically acceptable salt, hydrate or solvate thereof
wherein compounds of formula I are 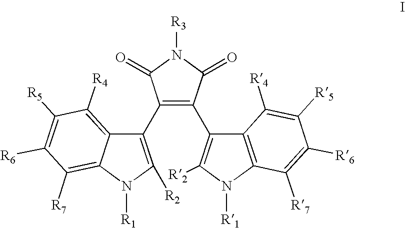
wherein
each of R₁ and R′₁, independently, is hydrogen, alkyl, haloalkyl, alkenyl, arylalkyl, alkoxyalkyl, hydroxyalkyl, aminoalkyl, monoalkylaminoalkyl, dialkylaminoalkyl, acylaminoalkyl, acyloxyalkyl, cyanoalkyl, amidinoalkyl, carboxyalkyl, alkoxycarbonylalkyl, aminocarbonylalkyl, or a group of the formula (a), (b) or (c) 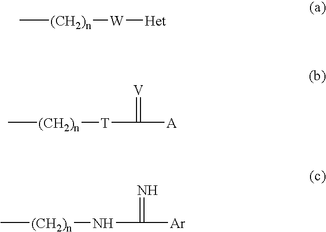
wherein Het signifies a heterocyclyl group; W signifies NH, S or a bond; T signifies NH or S; V signifies O, S. NH, or NCN; A signifies alkylthio, amino, monoalkylamino or dialkylamino; Ar signifies aryl;
each of R₂ and R′₂, independently, is hydrogen, alkyl, alkoxyalkyl, hydroxyalkyl, C₁-C₃alkylthio, S(O)C₁-C₃alkyl, CF₃;
or R₁ and R₂ form together —(CH2)ᵣ—X—CH₂— wherein r is 1, 2, or 3, and X is CHR₈ or NR₈ wherein R₈ is (CH₂)ₛR₉ wherein R₉ is hydrogen, hydroxy, alkoxy, amino, monoalkylamino, dialkylamino, trialkylamino, azido, acylamino, alkoxycarbonyl, cyano, amidino, or aminocarbonyl, and s is 0, 1, 2 or 3;
R₃ is hydrogen or CH₃CO;
each of R₄, R′₄, R₅, R′₅, R₆, R′₆, R₇ and R′₇, independently, is hydrogen, halogen, alkyl, hydroxy, alkoxy, —COO(C₁-C₃alkyl), CF₃, nitro, amino, acetylamino, monoalkylamino, dialkylamino, alkylthio, C₁-C₃alkylthio, or S(O)C₁-C₃alkyl; and
n is 1, 2, 3, 4, 5 or 6;
and compounds of formula II are 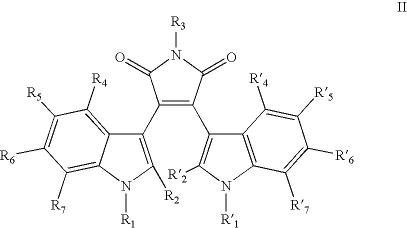
wherein
R₁ is a group of formula (d), (e) or (f) 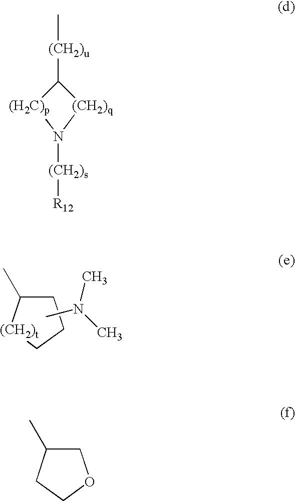
wherein each of p and q independently is 1, 2, 3, or 4;
s is 0, 1, 2 or 3;
t is 1 or 2;
u is 0 or 1; and
R₁₂ is hydrogen, alkyl, haloalkyl, cycloalkyl, acetyl, aryl, —CH(aryl)₂, amino, monoaikylamino, dialkylamino, guanidino, —C(═N(alkoxycarbonyl))NH(alkyoxy-carbonyl), amidino, hydroxy, carboxy, alkoxycarbonyl or heterocyclyl;
R′₁ is hydrogen, C₁-₄alkyl, aminoalkyl, monoalkylaminoalkyl, or dialkylaminoalkyl, each of R₂ and R′₂, independently, is hydrogen, alkyl, alkoxyalkyi, hydroxyalkyl, C₁-C₃alkylthio, S(O)C₁-C₃alkyl, CF₃;
R₃ is hydrogen or CH₃CO—; and
each of R₄, R′₄, R₅, R′₅, R₆, R′₆, R₇ and R′₇, independently, is hydrogen, halogen, alkyl, hydroxy, alkoxy, —COO(C₁-C₃alkyl), CF₃, nitro, amino, acetylamino, monoalkylamino, dialkylamino, alkylthio, C₁-C₃alkylthio, or S(O)C₁-C₃alkyl;
and compounds of formula III are 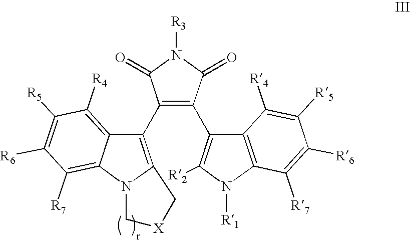
wherein
R′₁ is hydrogen, C₁-C₄alkyl, aminoalkyl, monoalkylaminoalkyl, or dialkylaminoalkyl;
R′₂ is hydrogen, alkyl, alkoxyalkyl, hydroxyalkyl, C₁-C₃alkyithio, S(O)C₁-C₃alkyl, CF₃ R₃ is hydrogen or CH₃CO—;
each of R₄, R′₄, R₅, R₆, R′₆, R₇ and R′₇, independently, is hydrogen, halogen, alkyl, hydroxy, alkoxy, —COO(C₁-C₃alkyl), CF₃, nitro, amino, acetylamino, monoalkylamino, dialkylamino, alkylthio, C₁-C₃alkylthio, or S(O)C₁-C₃alkyl;
X is CR₈R₉ wherein R₈ is (CH₂)ₛR₁₀ wherein R₉ is (CH₂)ₛR₁₁, each of R₁₀ and R₁₁, independently, is hydroxy, alkoxy, carboxy, acyloxy, amino, monoalkylamino, dialkylamino, trialkylamino, azido, acylamino, alkoxycarbonyl, cyano, amidino, or aminocarbonyl, and s is 0, 1, 2 or 3; and
r is 1, 2, or 3; and
and compounds of formula IV are 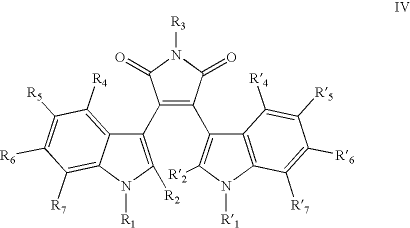
wherein
R₁ at is alkylglycose residue or a group of formula (g) or (h) 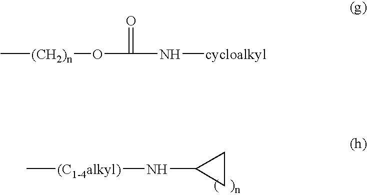
wherein n is 1, 2, 3, 4, 5 or 6;
R′₁ is hydrogen, C₁-C₄alkyl, cyclopropylmethyl, aminoalkyl, monoalkylaminoalkyl, or, dialkylaminoalkyl;
each of R₂ and R′₂, independently, is hydrogen, alkyl, alkoxyalkyl, hydroxyalkyl, C₁-C₃alkylthio, S(O)C₁-C₃alkyl, CF₃;
R₃ is hydrogen or CH₃CO—; and
each of R₄, R′₄, R₅, R′₅, R₆, R′₆, R₇ and R′₇, independently, is hydrogen, halogen, alkyl, hydroxy, alkoxy, —COO(C₁-C₃alkyl), CF₃, nitro, amino, acetylamino, monoalkylamino, dialkylamino, alkylthio, C₁-C₃alkylthio, or S(O)C₁-C₃alkyl.

2. A method according to claim 1 for the treatment or prevention of an autoimmune disease wherein the autoimmune diseases is selected from an inflammatory bowel disease amyotrophic lateral sclerosis; multiple sclerosis; rheumatoid arthritis and hepatitis C.

3. A method according to claim 1, for the treatment and prevention of organ or tissue transplant rejection or for the prevention of graft-versus-host disease.

4. A method according to claim 1 wherein the protein kinase C inhibitor is a compound of formula Ia, Ib, IIa, IIIa or a pharmaceutically acceptable salt, hydrate or solvate thereof.

5. A method according to claim 1 wherein the protein kinase C inhibitor is 3-(1-methyl-1H-indol-3-yl)-4-[1-{(1-pyridin-2-ylmethyl)-piperidin-4-yl}-1H-indol-3-yl]-pyrrole-2,5-dione, or 3-(1-methyl-1H-indol-3-yl)-4-[1-(piperidin-4-yl)-1H-indol-3-yl]-pyrrole-2,5-dione, or a pharmaceutically acceptable salt, hydrate or solvate thereof.

6. A pharmaceutical composition for use in the treatment and prevention of organ or tissue transplant rejection and for the prevention of graft-versus-host disease and/or of autoimmune diseases comprising a protein kinase C inhibitor of formula I, II, III or IV as defined in claim 1 or a pharmaceutically acceptable salt, hydrate or solvate thereof, together with one or more pharmaceutically acceptable diluents or carriers therefor.

7. A composition according to claim 6 wherein the protein kinase C inhibitor is a compound of formula Ia, Ib, IIa, IIIa or a pharmaceutically acceptable salt, hydrate or solvate thereof.

8. A composition according to claim 6 wherein the protein kinase C inhibitor is 3-(1-methyl-1H-indol-3-yl)-4-[1-{(1-pyridin-2-ylmethyl)-piperidin-4-yl}-1H-indol-3-yl]-pyrrole-2,5-dione or 3-(1-methyl-1H-indol-3-yl)-4-[1-(piperidin-4-yl)-1H-indol-3-yl]-pyrrole-2,5-dione, or a pharmaceutically acceptable salt, hydrate or solvate thereof.

9. A pharmaceutical combination comprising a) a protein kinase C inhibitor of formula I, II, III or IV as defined in claim 1, or a pharmaceutically acceptable salt, hydrate or solvate thereof, and b) at least one second agent selected from an immunosuppressant and immunomodulatory drug.

10. A pharmaceutical combination comprising a) a protein kinase C inhibitor of formula Ia, Ib, IIa, or IIIa as defined in claim 1, or a pharmaceutically acceptable salt, hydrate or solvate thereof and b) at least one second agent selected from an immunosuppressant and immunomodulatory drug.

11. (canceled)

12. A method according to claim 2 wherein the protein kinase C inhibitor is 3-(1-methyl-1H-indol-3-yl)-4-[1-{(1-pyridin-2-ylmethyl)-piperidin-4-yl}-1H-indol-3-yl]-pyrrole-2,5-dione, or 3-(1-methyl-1H-indol-3-yl)-4-[1-(piperidin-4-yl)-1H-indol-3-yl]-pyrrole-2,5-dione, or a pharmaceutically acceptable salt, hydrate or solvate thereof.

13. A method according to claim 3 wherein the protein kinase C inhibitor is 3-(1-methyl-1H-indol-3-yl)-4-[1-{(1-pyridin-2-ylmethyl)-piperidin-4-yl}-1H-indol-3-yl]-pyrrole-2,5-dione, or 3-(1-methyl-1H-indol-3-yl)-4-[1-(piperidin-4-yl)-1H-indol-3-yl]-pyrrole-2,5-dione, or a pharmaceutically acceptable salt, hydrate or solvate thereof.

14. A pharmaceutical combination according to claim 10 wherein a) is 3-(1-methyl-1H-indol-3-yl)-4-[1-{(1-pyridin-2-ylmethyl)-piperidin-4-yl}-1H-indol-3-yl]-pyrrole-2,5-dione or 3-(1-methyl-1H-indol-3-yl)-4-[1-(piperidin-4-yl)-1H-indol-3-yl]-pyrrole-2,5-dione.
